(19) United States
(12) Patent Application Publication
(10) Pub. No.: US 2024/0277799 A1
(43) Pub. Date: Aug. 22, 2024

(54) Translational Activators, Including Methods of Discovery and Uses Thereof

(71) Applicant: The Board of Trustees of the Leland Stanford Junior University, Stanford, CA (US)

(72) Inventors: Maria Barna, Stanford, CA (US); Travis Lantz, Stanford, CA (US)

(73) Assignee: The Board of Trustees of the Leland Stanford Junior University, Stanford, CA (US)

(21) Appl. No.: 18/582,265

(22) Filed: Feb. 20, 2024

Related U.S. Application Data
(60) Provisional application No. 63/485,798, filed on Feb. 17, 2023.

(51) Int. Cl. A61K 38/14; G01N 33/50; G01N 33/68

(52) U.S. Cl. CPC A61K 38/14; G01N 33/5008; G01N 33/6893; G01N 2800/22; G01N 2800/7057

(57) ABSTRACT

Translational activators are disclosed along with uses thereof and methods to discover translational activators. Many embodiments provide methods to treat diseases and disorders caused by global downregulation of protein synthesis, including (but not limited to) ribosomopathies and neurodegenerative disorders. Certain embodiments are directed to uses of translational activators for the manufacture of medicaments to treat diseases and disorders caused by global downregulation of protein synthesis, and further embodiments are directed to methods of discovering translational activators.

### CROSS REFERENCE TO RELATED APPLICATIONS

This application claims priority to U.S. Provisional Application Ser. No. 63/485,798, entitled “Translational Activators, Including Methods of Discovery and Uses Thereof,” filed Feb. 17, 2023, the disclosure of which is incorporated herein by reference in their entirety.

### FIELD OF THE INVENTION

The present invention relates to translational activators. More specifically, the present invention relates to methods and systems to identify activators of protein translation and treating medical disorders related to global downregulation of protein synthesis with translational activators.

### BACKGROUND

Cells are characterizable by a level of global protein synthesis that provides structural and enzymatic activity of each cell. The amount of global protein synthesis of a cell is regulated by a complex interaction of numerous signaling pathways involving each level of the molecular central dogma (i.e., DNA transcription into RNA and RNA translation into proteins). Dysregulation of molecular processes leads to tissue-specific phenotypes in all tissues, from limb to blood to brain. Ribosomopathies, such as Diamond-Blackfan Anemia (DBA), Schwachman-Diamond syndrome, 5q minus (5q−) syndrome, X-linked Dyskeratosis Congenita, cartilage-hair hypoplasia, Treacher-Collins syndrome (TCS), Bowen-Conradi syndrome, North American Indian childhood cirrhosis, and several neurological diseases, including amyotrophic lateral sclerosis (ALS), Vanishing White Matter Syndrome, Schizophrenia, Charcot Marie Tooth Syndrome, are considered to be caused by disruption of these molecular processes resulting in insufficient global protein synthesis. As a specific example, DBA is a fatal disorder in which a heterozygous mutation in one of 20 distinct ribosomal proteins presents with tissue-specific red blood cell aplasia leading to bone marrow defects and congenital birth defects. Corticosteroids are the only FDA-approved drug therapy, however only 50%-75% of DBA patients respond well to corticosteroids and chronic use of such drugs can have sever adverse effects including: immunosuppression, inhibition of bone growth, and osteoporosis. Therefore, there is a great need for novel therapeutics in treatment of DBA and other diseases and disorders caused by global downregulation of protein synthesis.

### SUMMARY OF THE INVENTION

This summary is meant to provide some examples and is not intended to be limiting of the scope of the invention in any way. For example, any feature included in an example of this summary is not required by the claims, unless the claims explicitly recite the features. Various features and steps as described elsewhere in this disclosure may be included in the examples summarized here, and the features and steps described here and elsewhere can be combined in a variety of ways.

In some implementations, a method is for treating a medical disorder characterizable by insufficient global protein synthesis.

In some implementations, the treatment method comprises administering a pharmaceutical composition comprising a translational activator compound to an individual.

In some implementations, the individual has a medical disorder characterizable by insufficient global protein synthesis.

In some implementations, the translational activator is a macrolide.

In some implementations, the macrolide is telithromycin.

In some implementations, the macrolide is erythromycin.

In some implementations, the macrolide is azithromycin.

In some implementations, the macrolide is solithromycin.

In some implementations, the medical disorder is characterizable by a genetic mutation in a ribosomal protein.

In some implementations, the ribosomal protein is RPS6, RPS19, RPS26, RPL5, or RPL11.

In some implementations, the medical disorder is characterizable by a genetic mutation in SBDS ribosome maturation factor (SBDS), dyskerin pseudouridine synthase 1 (DKC1), RNA component of mitochondrial RNA processing endoribonuclease (RMRP), treacle ribosome biogenesis factor 1 (TCOF1), EMG1 N1-specific pseudouridine methyltransferase (EMG1), UTP4 small subunit processome component (UTP4), or an aminoacyl-tRNA synthetase.

In some implementations, the medical disorder is selected from the group consisting of: Diamond-Blackfan Anemia (DBA), Schwachman-Diamond syndrome, 5q minus (5q−) syndrome, X-linked Dyskeratosis Congenita, cartilage-hair hypoplasia, Treacher-Collins syndrome (TCS), Bowen-Conradi syndrome, North American Indian childhood cirrhosis, amyotrophic lateral sclerosis (ALS), Vanishing White Matter Syndrome, Schizophrenia, and Charcot Marie Tooth Syndrome.

In some implementations, the medical disorder is Diamond-Blackfan Anemia (DBA).

In some implementations, diagnosing the individual with the medical disorder characterizable by insufficient global protein synthesis.

In some implementations, diagnosing the individual comprises assessing a tissue sample of the individual for insufficient global protein synthesis.

In some implementations, diagnosing the individual comprises assessing whether a translational activator compound rescues insufficient global protein synthesis.

In some implementations, diagnosing the individual comprises assessing a genetic sample of the individual to determine the presence of a genetic mutation within a ribosomal protein, SBDS ribosome maturation factor (SBDS), dyskerin pseudouridine synthase 1 (DKC1), RNA component of mitochondrial RNA processing endoribonuclease (RMRP), treacle ribosome biogenesis factor 1 (TCOF1), EMG1 N1-specific pseudouridine methyltransferase (EMG1), UTP4 small subunit processome component (UTP4), or an aminoacyl-tRNA synthetase.

In some implementations, a method screens for translational activator compounds.

In some implementations, the screening method comprises culturing a collection of cells characterizable by insufficient global protein synthesis.

In some implementations, the screening method comprises contacting the collection of cells with a compound.

In some implementations, the screening method comprises measuring global protein synthesis of the collection of cells.

In some implementations, the culture of cells comprises cells with a decrease in expression of a ribosomal protein.

In some implementations, wherein the culture of cells comprises cells with a decrease in expression of a ribosomal RNA.

In some implementations, measuring global protein synthesis comprises measuring the incorporation of a detectable marker within nascently synthesized proteins.

In some implementations, the detectable marker comprises L-Azidohomoalanine, L-homopropargylglycine, O-propargyl-puromycin, or a radioactive amino acid.

Other features and advantages of the present invention will become apparent from the following detailed description, taken in conjunction with the accompanying drawings which illustrate, by way of example, the principles of the invention.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIGS. 1A-1B provide data showing that a small increase in global protein synthesis has the ability to rescue a ribosomopathy phenotype.

FIGS. 2A-2D provide data and illustrate a schematic of an example of a method to screen compounds for increasing protein translation in accordance with various embodiments.

FIG. 3 provides chemical structures of various macrolides that have an ability to increase protein translation in accordance with various embodiments.

FIGS. 4A-4D provide data showing the ability of various macrolides to increase global protein synthesis.

FIG. 5 provides data showing cellular toxicity of various macrolides.

FIG. 6 provides a flow chart of an example of method to treat an individual with a translational activator in accordance with various embodiments.

### DETAILED DESCRIPTION

Turning now to the drawings and description, methods to discover translational activators, compounds translational activators, and methods of treatments using translational activators are disclosed. In many embodiments, a translational activator is administered to an individual to treat diseases and disorders caused by global downregulation of protein synthesis, such as (for example) ribosomopathies. Certain embodiments are directed to the manufacture of medicaments and formulations comprising a translational activator for the treatment of diseases and disorders caused by global downregulation of protein synthesis. Furthermore, several embodiments are directed to methods to screen compounds for identifying translational activators capable of increasing global translation.

Ribosomopathies are a class of congenital human disorders caused by mutations in essential ribosomal proteins (RPs) or ribosome biogenesis factors which manifest themselves with exquisite tissue specificity. In the 1930s, Josephs, Diamond, and Blackfan described infants with an unusual, highly selective congenital defect in hematopoiesis that presented with pure red blood cell aplasia without affecting other hematopoietic lineages, known as Diamond Blackfan anemia (DBA). Since then, DBA has been found to occur in ˜1:100,000 live births with newborns harboring a heterozygous mutation in one of 20 distinct RPs, leading to bone marrow failure and a spectrum of birth defects. Examples of congenital birth defects include craniofacial abnormalities as well as defects in digit and limb development. Bone marrow failure, however, is the leading cause of mortality in DBA and typically results in premature fatality. Currently, the standard treatment for DBA is administration of corticosteroids and frequent blood transfusions and/or hematopoietic stem cell transplants. Despite these treatment efforts, the majority of patients succumb to bone marrow failure. Corticosteroids, such as prednisone, are the only FDA-approved small molecule therapy, however, only 50%-75% of patients respond well to the corticosteroids and chronic use of the corticosteroids can result in adverse effects such as immunosuppression, inhibition of bone growth, and osteoporosis.

As ribosomes are universally required for translation, the mechanisms underlying tissue-specific defects in DBA, and other diseases and disorders caused by global downregulation of protein synthesis, have remained mysterious. The few studies that have looked at translational effects have mostly relied on ex vivo cell culture. While valuable, these systems do not accurately pinpoint the cellular context necessary to understand the complex phenotypes observed in ribosomopathies and further do not fully exemplify translation perturbation in an in vivo context.

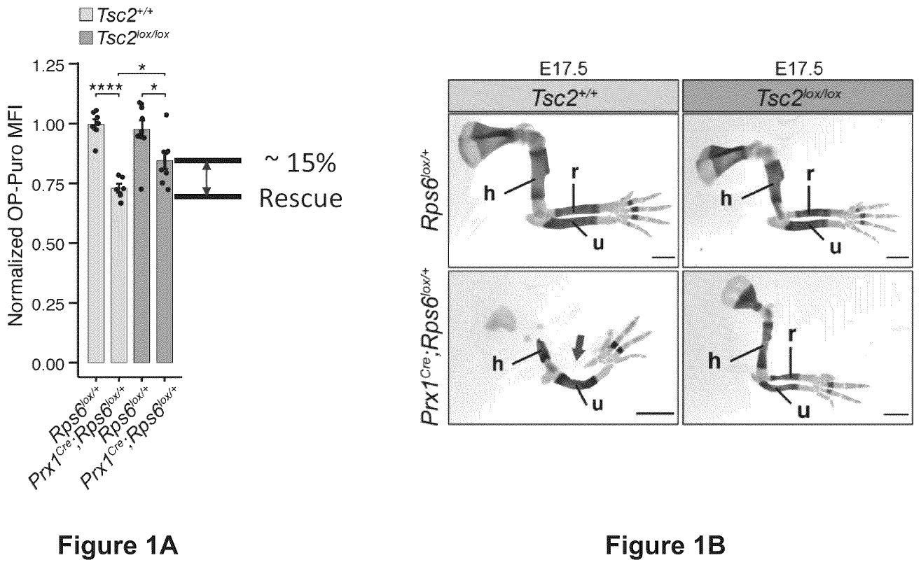

To understand the role of global translation in a cellular model of a ribosomopathy (e.g., DBA), a genetic mouse model of DBA was further genetically modified to rescue global transcription. Mice with haploinsufficient Rps6 have insufficient protein synthesis and as a result display limb morphogenesis abnormalities during development, mimicking some of the phenotypes observed in DBA. To rescue protein synthesis in the DBA mice, Tsc2, whose product is a regulator of mTORC1, was controllably deleted, resulting in an increase of mTOR pathway activity and an increase of global protein synthesis. As can be seen in FIG. 1A, deletion of Tsc2 resulted in a 15% increase of global protein synthesis in the DBA mouse model. The increase in global protein synthesis results was concurrent with restoration of normal limb development in the DBA mice (FIG. 1B). These results show that a 15% increase global protein synthesis rescues a common DBA phenotype. These results indicate that compound-based activators of global protein translation have the potential to rescue diseases and disorders caused by global downregulation of protein synthesis phenotypes. With these results, it was a goal of the current application to identify exogenous translational activators that can be used to treat diseases and disorders related to global downregulation of protein synthesis, such as ribosomopathies and certain neurodegenerative disorders.

### Identification and Discovery of Translational Activators

Several embodiments of the disclosure are directed to methods of discovering translational activators. In many embodiments, translational activators can be discovered in a high throughput manner. Several embodiments are also directed orthologous confirmation of the rescue ability of identified transactional activators.

Generally, a translational activator is a compound that when contacted with a cell, increases the protein synthesis in some manner. In some embodiments, a translational activator increases global protein synthesis. In some embodiments, a translational activator increases global protein synthesis in cells that have a protein synthesis insufficiency. In some embodiments, a translational activator increases global protein synthesis in cells that are healthy or other was sufficient in protein synthesis. In some embodiments, a translational activator compound is a small molecule, a macromolecule, a biologic, or any other chemical or biochemical entity that increases protein synthesis when contacting a cell.

To screen for translational activators, a variety of methodologies can be utilized in accordance with various embodiments of the disclosure. Generally, a compound is contacted with a collection of cells and the protein synthesis is monitored. In some embodiments, the collection of cells has a protein synthesis insufficiency. In some embodiments, the cells are healthy or other was sufficient in protein synthesis. Various readouts can be utilized that look for a change in phenotype. In some embodiments, a phenotypic screen can look for nascent protein synthesis. To detect nascent protein synthesis, a detectable marker that is incorporated into a nascent polypeptide can be assessed, such detectable markers include (but are not limited to) L-Azidohomoalanine, L-homopropargylglycine, O-propargyl-puromycin, and radioactive amino acids (e.g., ³⁵S-methionine). Alternative methods to detect nascent protein synthesis include (but are not limited to) mass spectrometry, sequencing or labelling of actively transcribed mRNA (e.g., by fractionation of polyribosomes), ribosome footprinting and/or quantification. For more on phenotypic screens of protein synthesis, see, e.g., S. Iwasaki and T. T. Ignolia, Trends Biochem Sci. 2017 August; 42(8):612-624, the disclosure of which is incorporated herein by reference.

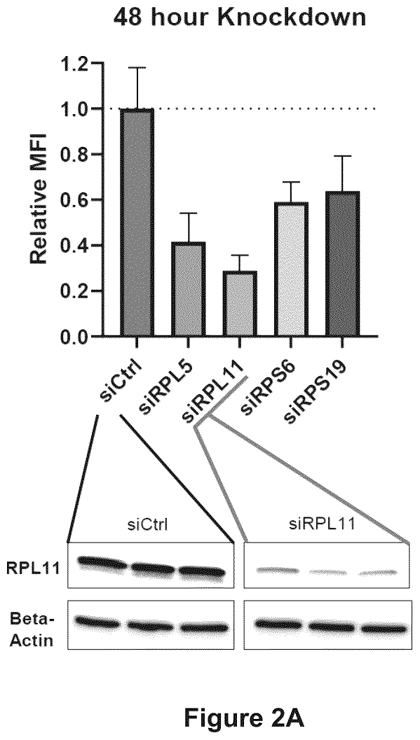

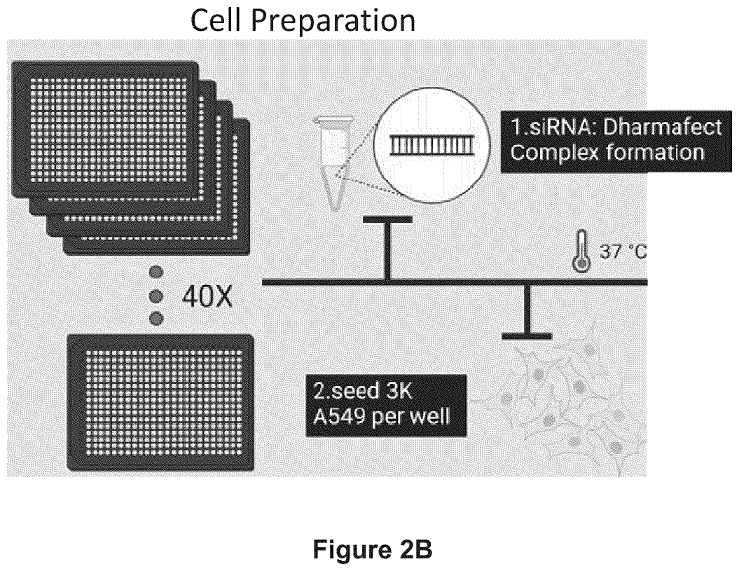

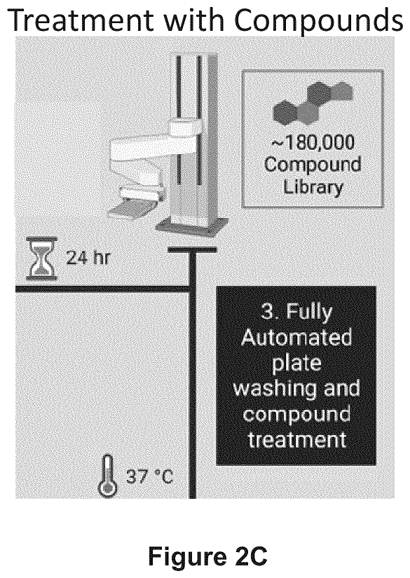

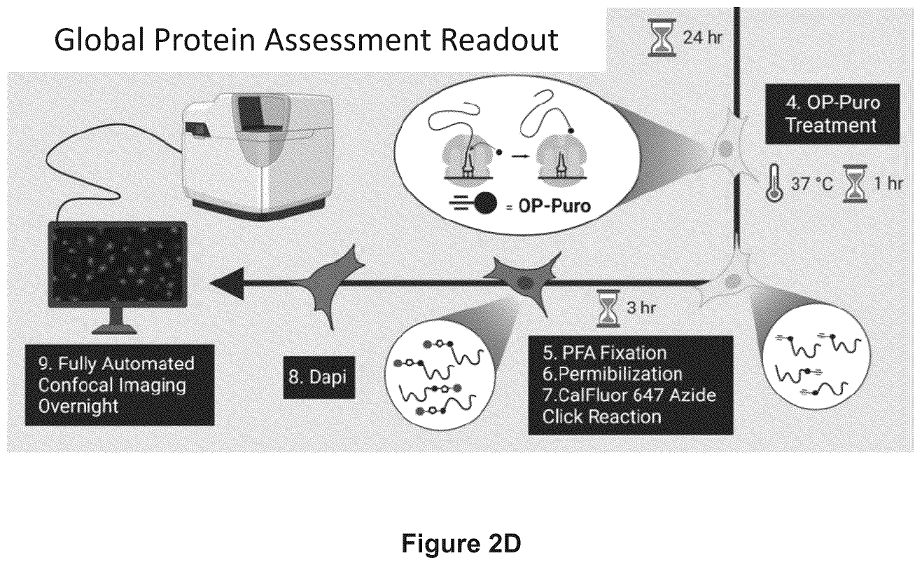

FIGS. 2A-2D provide data and a schematic of an example of a screening method to discover and/or identify translational activators. In this example, cells with decreased global protein synthesis via small interfering RNA (siRNA) knockdown of an RP are cultured within a multi-well format. Each well of cultured cells is contacted with a compound and then examined for global protein synthesis. Compounds that increased global protein synthesis were considered a putative translational activator for further examination.

Provided in FIG. 2A are data results of screening cells to identify a siRNA construct that effectively knocked down expression of a RP. In this experiment, A549 cells were utilized because they have a wild-type p53 sequence (e.g., no neoplasm-related p53 mutations), which is a major regulator and functional component in global translation synthesis. As can be seen in the results, each of the various siRNA constructs knocked down expression of an RP target.

An example of a 3-day screening method to assess compounds on global protein synthesis is provided in FIGS. 2B-2D. To begin, cells are prepared (FIG. 2B). In some embodiments, cells having decreased global protein translation are cultured. It should be understood that any cells can be assessed and it is not required that the cells have decreased global protein translation, but cells with decreased global protein translation may improve identification of translational activators. In some embodiments, the cells are known to have a wild-type p53 sequence, which can reduce cofounding factors. Culturing of cells can be in plates, tubes, flasks, and/or any other appropriate container. High throughput methods can use multi-well plates (e.g., 96-well, 384-well, 1536-well, etc.) or any other means for dividing a cell culture into multiple populations.

In some embodiments, the cells that are cultured are manipulated to decrease global translation. In some embodiments, cells are manipulated by reducing expression of or inhibiting functionality of a ribosomal subunit. Examples of ribosomal subunits that can be targeted include (but are not limited to) RPL5, RPL11, RPS6, and RPS19, each of which are involved in various ribosomopathies. One method to reduce ribosomal subunit expression is knockdown via antisense and/or RNA interference (RNAi) technology. As shown in FIG. 2A, siRNAs for RPL5, RPL11, RPS6, and RPS19 were each capable to knockdown protein levels of their target. The example method within FIG. 2B utilizes reverse transfection in which double-stranded siRNA complexes are preformed and transfected into the cells, which are then cultured.

Upon preparing the cells, the cells are then treated with compounds (FIG. 2C). In some embodiments, cultured cells are contacted with a compound to assess their effect on global protein translation. Compounds that can be utilized for assessment include (but are not limited to) small molecules, macromolecules, biologics, or any other chemical or biochemical entities. In certain embodiments, a culture of cells is contacted with singular compound at certain concentration. In some embodiments, a culture of cells is contacted with multiple compounds (concurrently or sequentially). Various embodiments provide combinations of multiple or individual compounds to each culture. In some embodiments, a pooling strategy utilized to reduce the number of individual cultures necessary to screen each compound. Such pooling can be of any relevant dimension (e.g., 2D, 3D, 4D, 5D, 6D, etc.). If using a pooling strategy, hits can be further assessed to delineate which compound (or combination of compounds) yield the beneficial effect. Compounds can be assessed at a single concentration, or multiple concentrations. Incubation time (e.g., the length of time prior to exchange of media) can be any appropriate time, from a couple of minutes to hours to days. For high-throughput assessments, multiple cell cultures (e.g., multiple wells) can be assessed concurrently, each cell culture provided with the same conditions except for the compound (or concentration thereof) utilized to contact the cells of the culture.

After a period of incubating the cell culture with a compound, global protein production can be assessed (FIG. 2D). Any readout for measuring protein synthesis can be utilized. In some embodiments, the cell culture is contacted with a detectable marker for detecting nascent protein synthesis. Examples of detectable markers include L-Azidohomoalanine, L-homopropargylglycine, O-propargyl-puromycin, and radioactive amino acids. In the illustrated example of FIG. 2D, O-propargyl-puromycin (OPP or OP-Puro) is utilized, which incorporates into the C-terminus of nascently synthesized polypeptides. OPP can be detected via click-chemistry in which the alkyne of OPP can be reacted with a probe to enable detection. Generally, any probe can be utilized such as (for example) a fluorescent probe. Fluorescence of the probe can be measured via a plate reader, microscopy, flow cytometry, and/or any other relevant mechanism to identify global protein production. For high-throughput assessments, fluorescence of multiple wells of cell culture can be assessed concurrently, which can be achieved using a plate reader or other device capable of measuring fluorescence of multiple wells.

Various metrics can be utilized for assessment. For example, when using a plate reader, overall fluorescence of a cultured and/or individual cellular fluorescence within a cultured well can be assessed. In some embodiments, if overall fluorescence intensity of a well is greater than a threshold, then the compound is considered a putative translational activator. In some embodiments, if a percentage of cells (e.g., 50% of cells) within the well have fluorescence intensity over a threshold, the compound is considered a putative translational activator. In some embodiments, the determination of a hit is compared to a control (e.g., untreated, or mock-treated) well. In some embodiments, the determination of a hit is determined by comparing fluorescence of a well at an initial timepoint and then an increase of fluorescence at one or more later timepoints.

The method of FIGS. 2B-2D may be repeated multiple times to screen additional compounds. For example, certain embodiments may perform one process to identify a pilot molecule, followed by additional rounds to find additional molecules with similar structure. Alternatively, additional rounds can be used to screen additional doses or concentrations of the compound. Identified compounds can be confirmed via orthologous assessment.

### Macrolides as Translational Activators

A number of compounds have been identified via screening that increase nascent protein synthesis. Interestingly, a number of macrolides were found to increase global protein synthesis. Macrolides are a class of compounds with a large macrocyclic lactone, having greater than 8 members. The macrocycle may contain amino nitrogen, amide nitrogen, an oxazole ring, or a thiazole ring. One or more deoxy sugars (e.g., cladinose or desosamine) are often attached to the lactone. Macrolides include telithromycin, erythromycin, azithromycin, solithromycin, clarithromycin, roxithromycin, carbomycin A, spiramycin, and other molecules. Many macrolides have antibiotic properties and several are approved by various jurisdictions for use as drug to treat various bacterial and/or parasitic infections.

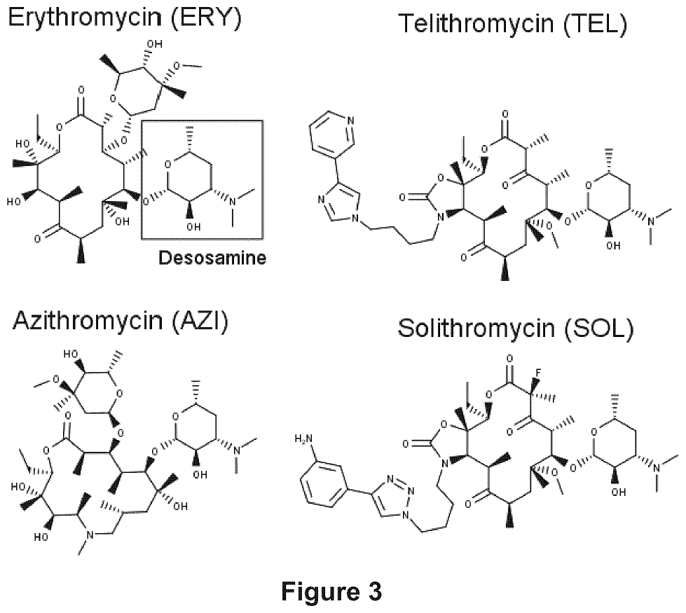

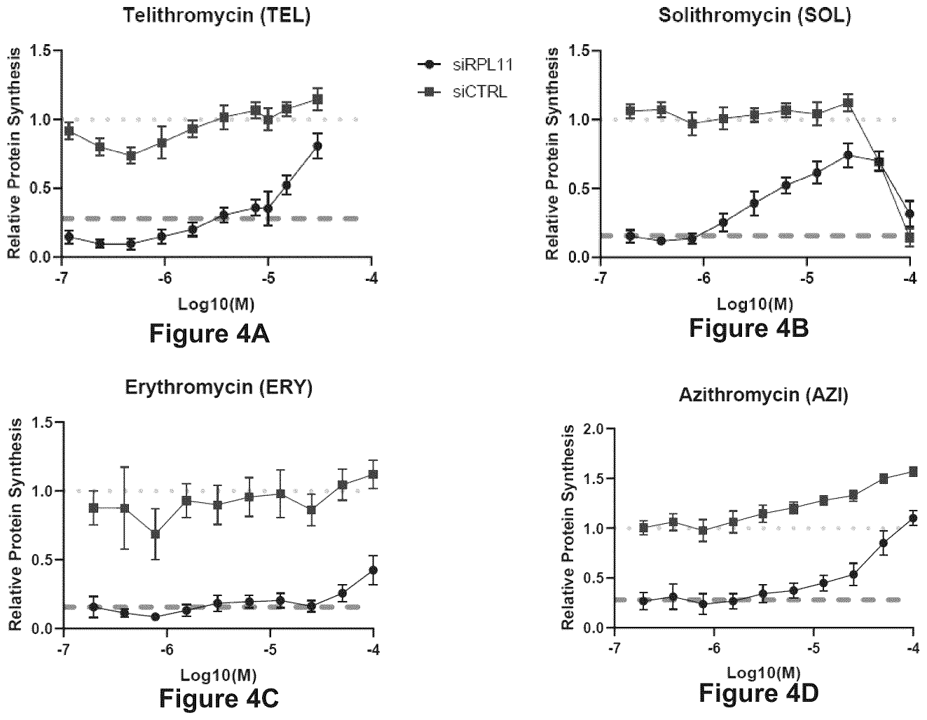

FIG. 3 provides structural diagrams of telithromycin, erythromycin, azithromycin, and solithromycin, each of which comprising a large macrocyclic lactone with attached deoxy sugars. These four compounds were further assessed for their ability to increase global translation. In these experiments, A459 cells transfected with siRNA targeting RPL11 (siRPL11) representing cells with insufficient global protein synthesis and A459 cells transfected with a control siRNA (siCTRL) representing cells with normal global synthesis were each subsequently treated with erythromycin, azithromycin, and solithromycin (FIGS. 4A-4D). Each of these macrolides had some ability to increase protein synthesis in the cells having insufficient global protein synthesis. Specifically, erythromycin increased protein synthesis at high concentrations in protein synthesis insufficient cells. Telithromycin, solithromycin, and azithromycin were able to increase protein synthesis at 10-fold lower concentrations as compared to erythromycin. Furthermore, azithromycin increased protein synthesis in control cells as well.

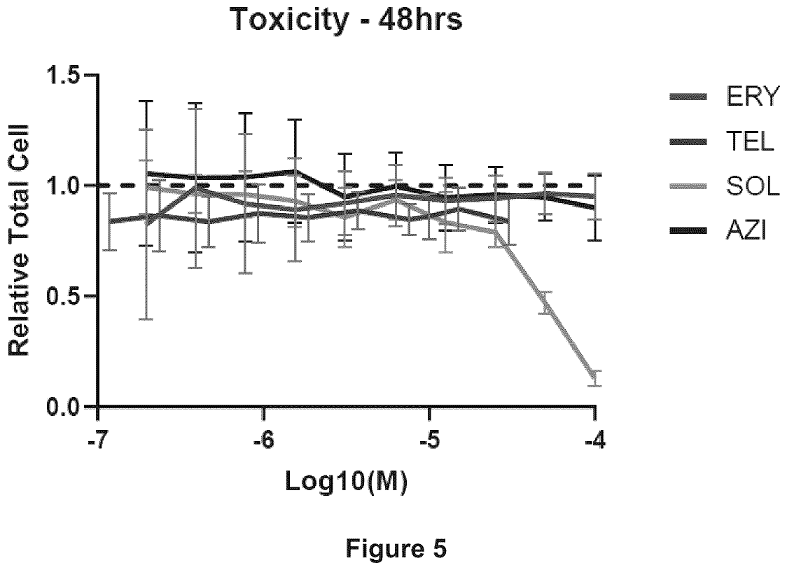

Turning to FIG. 5, cellular toxicity data of telithromycin, erythromycin, azithromycin, and solithromycin are provided. Specifically, FIG. 5 illustrates relative total cell count is plotted against the concentration provided. As indicated, these macrolides show little to no toxicity at concentrations that improve protein synthesis.

These data within FIGS. 4A-4D and FIG. 5 highlight the ability of macrolides to be used in the treatment medical disorders that are characterizable insufficient protein synthesis. Insufficient protein synthesis is to mean that a disorder is characterizable by having a decrease in cellular global protein synthesis, as would be compared to an individual unaffected by the disorder. Accordingly, macrolides are useful for treatment of ribosomopathies and some neurodegenerative disorders. Specifically, macrolides are useful in the treatment of Diamond-Blackfan Anemia (DBA), Schwachman-Diamond syndrome, 5q minus (5q−) syndrome, X-linked Dyskeratosis Congenita, cartilage-hair hypoplasia, Treacher-Collins syndrome (TCS), Bowen-Conradi syndrome, North American Indian childhood cirrhosis, amyotrophic lateral sclerosis (ALS), Vanishing White Matter Syndrome, Schizophrenia, Charcot Marie Tooth Syndrome, and various other disorders characterized by having global protein synthesis insufficiency. In some embodiments, global protein synthesis insufficiency is characterized by determining that the individual has a mutation in a ribosomal protein (especially RPS6, RPS14, RPS19, RPS26, RPL5, and RPL11), SBDS ribosome maturation factor (SBDS), dyskerin pseudouridine synthase 1 (DKC1), RNA component of mitochondrial RNA processing endoribonuclease (RMRP), treacle ribosome biogenesis factor 1 (TCOF1), EMG1 N1-specific pseudouridine methyltransferase (EMG1), UTP4 small subunit processome component (UTP4), or an aminoacyl-tRNA synthetase.

### Pharmaceutical Compositions Comprising Translational Activators

In certain embodiments, the present disclosure provides pharmaceutical compositions comprising one or more translational activators and/or a salt thereof. In certain such embodiments, the pharmaceutical composition comprises a suitable pharmaceutically acceptable diluent or carrier. In certain embodiments, a pharmaceutical composition comprises a sterile saline solution and one or more translational activators. In certain embodiments, the sterile saline is pharmaceutical grade saline. In certain embodiments, a pharmaceutical composition comprises sterile water and one or more translational activators. In certain embodiments, the water is pharmaceutical grade water. In certain embodiments, a pharmaceutical composition comprises phosphate-buffered saline (PBS) and one or more translational activators. In certain embodiments, the PBS is pharmaceutical grade PBS.

In certain embodiments, pharmaceutical compositions comprise one or more translational activators and one or more excipients. In certain such embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, translational activators may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising a translational activator encompass any pharmaceutically acceptable salts of the translational activator, esters of the translational activator, or salts of such esters. In certain embodiments, pharmaceutical compositions comprising a translational activator encompass any pharmaceutically acceptable salts of the translational activator. In certain embodiments, pharmaceutical compositions comprising one or more translational activators, upon administration to an animal, including a human, are capable of providing (directly or indirectly) the biologically active metabolite or residue thereof. Accordingly, for example, the disclosure is also drawn to pharmaceutically acceptable salts of translational activators, prodrugs, pharmaceutically acceptable salts of such prodrugs, and other bioequivalents. Suitable pharmaceutically acceptable salts include, but are not limited to, sodium and potassium salts. In certain embodiments, prodrugs comprise one or more conjugate group attached to a translational activator, wherein the conjugate group is cleaved by endogenous enzymes, including (but not limited to) esterases, cytochrome p450s, etc., within the body.

In certain embodiments, a pharmaceutical composition comprises a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethyl sulfoxide (DMSO) are used.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose. In certain embodiments, dimethyl sulfoxide (DMSO) is utilized as a co-solvent. In certain embodiments, cremophor (or cremophor EL) is utilized as a co-solvent.

In certain embodiments, pharmaceutical compositions comprise one or more compounds that increase bioavailability. For example, 2-hydroxypropyl-beta-cyclodextrin can be utilized in pharmaceutical compositions and may increase bioavailability. In certain embodiment, DMSO, cremophor and 2-hydroxypropyl-beta-cyclodextrin, and mixtures thereof, may be utilized to increase bioavailability of various translational activators.

In certain embodiments, pharmaceutical compositions are prepared for oral administration. In certain embodiments, pharmaceutical compositions are prepared for buccal administration. In certain embodiments, pharmaceutical compositions are prepared for aerosol or nebulizer administration. In certain embodiments, a pharmaceutical composition is prepared for administration by injection (e.g., intravenous, subcutaneous, intramuscular, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

In certain embodiments, a pharmaceutical composition is administered in a therapeutically effective amount as part of a course of treatment. As used in this context, to “treat” means to ameliorate or prevent at least one symptom of the disorder to be treated or to provide a beneficial physiological effect. A therapeutically effective amount can be an amount sufficient to prevent, reduce, ameliorate or eliminate the symptoms of diseases or pathological conditions susceptible to such treatment. In certain embodiments, a therapeutically effective amount is an amount sufficient to inhibit virus replication.

Dosage, toxicity and therapeutic efficacy of a pharmaceutical composition can be determined, e.g., by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

Data obtained from cell culture assays or animal studies can be used in formulating a range of dosage for use in humans. If a pharmaceutical composition is provided systemically, the dosage of such compounds lies preferably within a range of circulating concentrations that include the ED₅₀ with little or no toxicity. The dosage may vary within this range depending upon the dosage form employed and the route of administration utilized. For any compound used in a method described herein, the therapeutically effective dose can be estimated initially from cell culture assays. A dose may be formulated in animal models to achieve a circulating plasma concentration or within the local environment to be treated in a range that includes the IC₅₀ (i.e., the concentration of the test compound that achieves a half-maximal inhibition of virus propagation) as determined in cell culture. Such information can be used to more accurately determine useful doses in humans. Levels in plasma may be measured, for example, by liquid chromatography coupled to mass spectrometry.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a pharmaceutical composition described herein can include a single treatment or a series of treatments. For example, several divided doses may be administered daily, one dose, or cyclic administration of the compounds to achieve the desired therapeutic result. A single small molecule compound may be administered, or combinations of various small molecule compounds may also be administered.

It is also possible to add agents that improve the solubility of pharmaceutical compositions. For example, a pharmaceutical composition can be formulated with one or more adjuvants and/or pharmaceutically acceptable carriers according to the selected route of administration. For oral applications, gelatin, flavoring agents, or coating material can be added. In general, for solutions or emulsions, carriers may include aqueous or alcoholic/aqueous solutions, emulsions or suspensions, including saline and buffered media. Parenteral vehicles can include sodium chloride and potassium chloride, among others. In addition, intravenous vehicles can include fluid and nutrient replenishers, electrolyte replenishers and the like.

Numerous coating agents can be used in accordance with various embodiments. In certain embodiments, the coating agent is one which acts as a coating agent in conventional delayed release oral formulations, including polymers for enteric coating. Examples include hypromellose phthalate (hydroxy propyl methyl cellulose phthalate; HPMCP); hydroxypropylcellulose (HPC; such as KLUCEL®); ethylcellulose (such as ETHOCEL®); and methacrylic acid and methyl methacrylate (MAA/MMA; such as EUDRAGIT®).

In certain embodiments, a pharmaceutical composition also includes at least one disintegrating agent, as well as diluent. In some embodiments, a disintegrating agent is a super disintegrant agent. One example of a diluent is a bulking agent such as a polyalcohol. In many embodiments, bulking agents and disintegrants are combined, such as, for example, PEARLITOL FLASH®, which is a ready to use mixture of mannitol and maize starch (mannitol/maize starch). In accordance with a number of embodiments, any polyalcohol bulking agent can be used when coupled with a disintegrant or a super disintegrant agent. Additional disintegrating agents include, but are not limited to, agar, calcium carbonate, maize starch, potato starch, tapioca starch, alginic acid, alginates, certain silicates, and sodium carbonate. Suitable super disintegrating agents include, but are not limited to crospovidone, croscarmellose sodium, AMBERLITE (Rohm and Haas, Philadelphia, Pa.), and sodium starch glycolate.

In certain embodiments, diluents are selected from the group consisting of mannitol powder, spray dried mannitol, microcrystalline cellulose, lactose, dicalcium phosphate, tricalcium phosphate, starch, pregelatinized starch, compressible sugars, silicified microcrystalline cellulose, and calcium carbonate.

In certain embodiments, a pharmaceutical composition further utilizes other components and excipients. For example, sweeteners, flavors, buffering agents, and flavor enhancers to make the dosage form more palatable. Sweeteners include, but are not limited to, fructose, sucrose, glucose, maltose, mannose, galactose, lactose, sucralose, saccharin, aspartame, acesulfame K, and neotame. Common flavoring agents and flavor enhancers that may be included in the formulations described herein include, but are not limited to, maltol, vanillin, ethyl vanillin, menthol, citric acid, fumaric acid, ethyl maltol and tartaric acid.

In certain embodiments, a pharmaceutical composition also includes a surfactant. In certain embodiments, surfactants are selected from the group consisting of Tween 80, sodium lauryl sulfate, and docusate sodium.

In certain embodiments, a pharmaceutical composition further utilizes a binder. In certain embodiments, binders are selected from the group consisting of povidone (PVP) K29/32, hydroxypropylcellulose (HPC), hydroxypropylmethylcellulose (HPMC), ethylcellulose (EC), corn starch, pregelatinized starch, gelatin, and sugar.

In certain embodiments, a pharmaceutical composition also includes a lubricant. In certain embodiments, lubricants are selected from the group consisting of magnesium stearate, stearic acid, sodium stearyl fumarate, calcium stearate, hydrogenated vegetable oil, mineral oil, polyethylene glycol, polyethylene glycol 4000-6000, talc, and glyceryl behenate.

Preservatives and other additives, like antimicrobial, antioxidant, chelating agents, and inert gases, can also be present. (See generally, Remington's Pharmaceutical Sciences, 16th Edition, Mack, (1980), the disclosure of which is incorporated herein by reference.)

### Modes of Treatments

As described herein, increasing global protein production can rescue medical disorders characterizable by insufficient global protein synthesis, including ribosomopathies and certain neurodegenerative disorders. Insufficient protein synthesis is to mean that a disorder is characterizable by having a decrease in cellular global protein synthesis, as would be compared to an individual unaffected by the disorder (or an average amount of global protein synthesis in individuals unaffected by the disorder). Translational activators, including macrolides, have the ability to increase global production, thus providing a treatment for these disorders. Thus, many embodiments are directed to methods of treating diseases and disorders characterizable by insufficient global protein synthesis, including ribosomopathies and some neurodegenerative disorders, including Diamond-Blackfan Anemia (DBA), Schwachman-Diamond syndrome, 5q minus (5q−) syndrome, X-linked Dyskeratosis Congenita, cartilage-hair hypoplasia, Treacher-Collins syndrome (TCS), Bowen-Conradi syndrome, North American Indian childhood cirrhosis, amyotrophic lateral sclerosis (ALS), Vanishing White Matter Syndrome, Schizophrenia, Charcot Marie Tooth Syndrome, and various other disorders characterized by having global protein synthesis insufficiency. In several embodiments, an individual having a disorder characterizable by insufficient global protein synthesis is administered a pharmaceutical composition comprising a translational activator. In some embodiments, an individual having a disorder characterizable by insufficient global protein synthesis is administered a pharmaceutical composition comprising a macrolide. In some embodiments, an individual having a disorder characterizable by insufficient global protein synthesis is administered a pharmaceutical composition comprising telithromycin. In some embodiments, an individual having a disorder characterizable by insufficient global protein synthesis is administered a pharmaceutical composition comprising erythromycin. In some embodiments, an individual having a disorder characterizable by insufficient global protein synthesis is administered a pharmaceutical composition comprising azithromycin. In some embodiments, an individual having a disorder characterizable by insufficient global protein synthesis is administered a pharmaceutical composition comprising solithromycin.

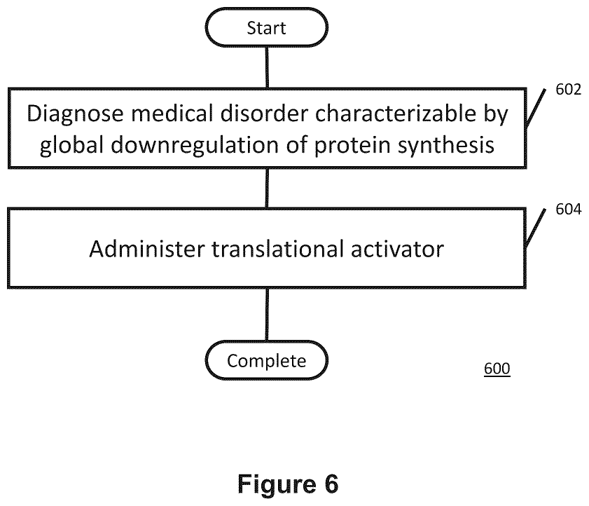

FIG. 6 provides a schematic of an example of a method to treat an individual for a disease or disorder caused by global downregulation of protein synthesis, in accordance with many embodiments. At 602 of Method 600, an individual is diagnosed with a medical disorder characterizable by global downregulation of protein synthesis. Various methods in the art are known to identify an individual and/or diagnose the individual with a disease or disorder caused by global downregulation of protein synthesis, including genetic, biochemical, physiological, metabolic, and/or any other relevant testing.

In some embodiments, a diagnosis comprises assessing a genetic sample of the individual for a genetic mutation. Genetic mutations within ribosomal proteins (especially RPS6, RPS19, RPS26, RPL5, and RPL11) SBDS ribosome maturation factor (SBDS), dyskerin pseudouridine synthase 1 (DKC1), RNA component of mitochondrial RNA processing endoribonuclease (RMRP), treacle ribosome biogenesis factor 1 (TCOF1), EMG1 N1-specific pseudouridine methyltransferase (EMG1), UTP4 small subunit processome component (UTP4), and aminoacyl-tRNA synthetases are known to give rise to medical disorders characterizable by insufficient global protein synthesis.

In some embodiments, a diagnosis comprises assessing a tissue sample of the individual for a genetic mutation. A tissue sample is to be understood to be any sample derived from the individual comprising cells of the individual. In some embodiments, the tissue sample is assessed for insufficient global protein synthesis. In some embodiments, the tissue sample is assessed to determine whether a translational activator compound rescues insufficient global protein synthesis. In some embodiments, if a translational activator compound rescues insufficient global protein synthesis, the individual is administered with said translational activator.

In certain embodiments, translational activators are administered as part of a course of treatment at 604. As used in this context, to “treat” means to ameliorate or prevent at least one symptom of the disease or disorder caused by global downregulation of protein synthesis to be treated or to provide a beneficial physiological effect. A translational activator can be a small molecule, a biologic (e.g., antibody, ligand, etc.), a nucleic acid (e.g., mRNA, siRNA, DNA, etc.), and/or any other therapy to increase global translation. Translational activators can be provided via any relevant means, including as an infusion, injection, orally, supositorally, and/or any other means of providing a translational activator to an individual.

A therapeutically effective amount can be an amount sufficient to prevent, reduce, ameliorate or eliminate the symptoms of diseases or pathological conditions susceptible to such treatment. In certain embodiments, a therapeutically effective amount is an amount sufficient to increase global protein synthesis.

It should be noted method 600 is not meant to be exhaustive of all possible embodiments of methods to treat an individual. Additional embodiments may include additional features, omit certain features, duplicate and/or replicate certain features, and/or perform certain features in a different order, including simultaneously.

### DOCTRINE OF EQUIVALENTS

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the foregoing examples and descriptions of various preferred embodiments of the present invention are merely illustrative of the invention as a whole, and that variations in the components or steps of the present invention may be made within the spirit and scope of the invention. Accordingly, the present invention is not limited to the specific embodiments described herein, but, rather, is defined by the scope of the appended claims.

What is claimed is:

1. A method of treating a medical disorder characterizable by insufficient global protein synthesis, comprising:
administering a pharmaceutical composition comprising a translational activator compound to an individual, wherein the individual has a medical disorder characterizable by insufficient global protein synthesis.

2. The method of claim 1, wherein the translational activator compound is a macrolide.

3. The method of claim 2, wherein the macrolide is telithromycin.

4. The method of claim 2, wherein the macrolide is erythromycin.

5. The method of claim 2, wherein the macrolide is azithromycin.

6. The method of claim 2, wherein the macrolide is solithromycin.

7. The method of claim 1, wherein the medical disorder is characterizable by a genetic mutation in a ribosomal protein.

8. The method of claim 7, wherein the ribosomal protein is RPS6, RPS19, RPS26, RPL5, or RPL11.

9. The method of claim 1, wherein the medical disorder is characterizable by a genetic mutation in SBDS ribosome maturation factor (SBDS), dyskerin pseudouridine synthase 1 (DKC1), RNA component of mitochondrial RNA processing endoribonuclease (RMRP), treacle ribosome biogenesis factor 1 (TCOF1), EMG1 N1-specific pseudouridine methyltransferase (EMG1), UTP4 small subunit processome component (UTP4), or an aminoacyl-tRNA synthetase.

10. The method of claim 1, wherein the medical disorder is selected from the group consisting of: Diamond-Blackfan Anemia (DBA), Schwachman-Diamond syndrome, 5q minus (5q−) syndrome, X-linked Dyskeratosis Congenita, cartilage-hair hypoplasia, Treacher-Collins syndrome (TCS), Bowen-Conradi syndrome, North American Indian childhood cirrhosis, amyotrophic lateral sclerosis (ALS), Vanishing White Matter Syndrome, Schizophrenia, and Charcot Marie Tooth Syndrome.

11. The method of claim 10, wherein the medical disorder is Diamond-Blackfan Anemia (DBA).

12. The method of claim 1 further comprising:
diagnosing the individual with the medical disorder characterizable by insufficient global protein synthesis.

13. The method of claim 12, wherein diagnosing the individual comprises assessing a tissue sample of the individual for insufficient global protein synthesis.

14. The method of claim 12, wherein diagnosing the individual comprises assessing whether a translational activator compound rescues insufficient global protein synthesis.

15. The method of claim 12, wherein diagnosing the individual comprises assessing a genetic sample of the individual to determine presence of a genetic mutation within a ribosomal protein, SBDS ribosome maturation factor (SBDS), dyskerin pseudouridine synthase 1 (DKC1), RNA component of mitochondrial RNA processing endoribonuclease (RMRP), treacle ribosome biogenesis factor 1 (TCOF1), EMG1 N1-specific pseudouridine methyltransferase (EMG1), UTP4 small subunit processome component (UTP4), or an aminoacyl-tRNA synthetase.

16. A method to screen for translational activator compounds, comprising:
culturing a collection of cells characterizable by insufficient global protein synthesis;
contacting the collection of cells with a compound; and
measuring global protein synthesis of the collection of cells.

17. The method of claim 16, wherein the collection of cells comprises cells with a decrease in expression of a ribosomal protein.

18. The method of claim 17, The method of claim 16, wherein the collection of cells comprises cells with a decrease in expression of a ribosomal RNA.

19. The method of claim 16, wherein measuring global protein synthesis comprises measuring incorporation of a detectable marker within nascently synthesized proteins.

20. The method of claim 19, wherein the detectable marker comprises L-Azidohomoalanine, L-homopropargylglycine, O-propargyl-puromycin, or a radioactive amino acid.
